(19) United States
(12) Patent Application Publication
(10) Pub. No.: US 2017/0020465 A1
(43) Pub. Date: Jan. 26, 2017

(54) OPERATING TABLE

(71) Applicant: IDTM GmbH, Castrop-Rauxel (DE)

(72) Inventor: Michael Friebe, Recklinghausen (DE)

(73) Assignee: IDTM GMBH, Castrop-Rauxel (DE)

(21) Appl. No.: 15/300,913

(22) PCT Filed: Mar. 31, 2015
(86) PCT No.: PCT/EP2015/057097

§ 371 Date: Sep. 30, 2016

(30) Foreign Application Priority Data
Mar. 31, 2014 (DE) 10 2014 104 529.7

(51) Int. Cl. A61B 6/04; A61G 13/02; A61G 13/10; A61B 6/03; A61B 6/00

(52) U.S. Cl. CPC A61B 6/0407; A61B 6/035; A61B 6/037; A61B 6/4452; A61B 6/54; A61G 13/10; A61G 13/02; A61G 2210/50

(57) ABSTRACT

An operating table including a stretcher for receiving a patient on a supporting surface of the stretcher, a detector which is arranged below the stretcher's supporting surface, an arcuate arm which is extendable from an area below the supporting surface at least partially into an area above the supporting surface, and an X-ray tube positioned in the arm.

### FIELD OF THE INVENTION

The invention relates to an operating table according to claim 1.

### PRIOR ART

Intraoperative X-ray scans and computer tomographies are used for various purposes. One possible purpose is the control of the navigation of instruments in the human brain. In doing so, the head is fixed and a computer tomography of the head is taken, for example, already after having introduced instruments into the head.

A further application is the use in back surgeries. After having fixed a marker to an extension of the vertebral column, the precise relative position of the marker, and thus the vertebral body, relative to implants or instruments can thus be detected by means of an X-ray scan or preferably a computer tomography. In this way, repeated X-raying is no longer required during the placement of implants. Also screws can be inserted more precisely with such systems. A navigation which is supported by intraoperative computer tomography provides advantages, for instance, in cases of serious scolioses.

A disadvantage of known devices for interoperatively taking X-ray images and computer tomographies, however, is their cumbersome handling. It is true that devices are known which consist of a torus with a removable part to facilitate entering a patient into the torus. Nevertheless, here as well, the effort to produce the computer tomography is enormous.

### DISCLOSURE OF THE INVENTION

The task of the invention is to propose an improved operating table, by means of which the prior art problems should be solved or alleviated.

The task is solved by an operating table according to claim 1. Typical further developments are included in the dependent claims.

A first aspect of the invention relates to an operating table including a stretcher for receiving a patient on a supporting surface of the stretcher, a detector which is arranged below the stretcher's supporting surface, an arcuate arm which is extendable from an area below the supporting surface at least partially into an area above the supporting surface, and an X-ray tube positioned in the arm.

Typical embodiments of the invention comprise a control device connected to the detector and the X-ray tube. The control device is typically configured to realize computer tomography by means of the X-ray tube and the detector. Typical embodiments comprise a monitor connected to the control device for displaying images of a computer tomography taken by the X-ray tube and the detector. Insofar as “the X-ray tube” or other features cited in singular are mentioned in this application, at least one X-ray tube or accordingly at least one feature is meant, that is, a plurality of X-ray tubes may be present, for example.

In typical embodiments, the arm comprises at least two or at least five X-ray tubes. This has the advantage that a potential computer tomography can be captured more quickly and precisely or sequential X-ray images can be taken more quickly and precisely. In typical embodiments, the operating table including the control device is configured to produce normal or conventional X-ray images or images in the radioscopic mode (fluoroscopy), to capture spiral computer tomography images or to prepare computer tomography images within a short period of time, e.g. a computer tomography image of a transverse plane in less than five seconds. In embodiments comprising a plurality of X-ray tubes in the arm, the X-ray tubes are typically arranged to be evenly distributed over the arm. In further embodiments, the X-ray tubes are arranged to be unevenly distributed over the arm in order to make more room for other devices. Typical embodiments of arms of embodiments of the invention comprise a monitor positioned in the arm. This has the advantage that a result can already be displayed for the operating surgeon immediately during the shooting. Arms without a monitor may possibly be more light-weight or smaller. Typical embodiments are not only suited for computer tomography but also for “normal” radioscopy. In the “normal” radioscopy as well, thus in taking X-ray images, two or more tubes, or one tube which is displaceable within the arm help to achieve several angles of vision. In this way, even simple or light-version computer tomographies may be captured which are mostly sufficient for navigation purposes.

Typical embodiments of operating tables according to the invention exhibit an arm having a radius of a maximum of 70 cm, a maximum of 50 cm or a maximum of 40 cm. This has the advantage of low space requirement. In this case, the radius is typically measured at an inner radius of the arm. Typical arms exhibit a circular shape or an elliptical shape or any other curved shape. Circular shapes provide the advantage that retracting the arm into a circular recess is space-saving.

In typical embodiments of operating tables according to the invention, the arm is displaceable relative to the table in a longitudinal direction of the table. The arm may, for example, be attached to a slide or actuator interacting with a linear guide. Typically, the arm is supported, for instance via an actuator, on a linear guide which is aligned in the longitudinal direction of the stretcher. In typical exemplary embodiments, the arm is kinematically coupled with the detector in the stretcher's longitudinal direction. The arm and detector may thereby be displaced simultaneously. The detector may be fastened, for example, to a longitudinally aligned linear guide together with the arm. Typical embodiments comprise actuators, for instance an actuator for extending the arm into an area above the supporting surface. Moreover, an actuator may be provided for displacing the arm lengthwise along the longitudinal direction of the table. Embodiments comprise actuators which are configured to perform both an extension of the arm and a linear translation of the arm along the operating table's longitudinal direction. By kinematically coupling the detector with the arm it is possible to displace the arm and the detector simultaneously along the longitudinal direction. This enables computer tomographies of transverse sections of a patient on the table in various axial heights of the patient. Typically, actuators for driving the arm are connected to the control device so as to establish and assemble computer tomographies of several levels.

Typical embodiments of operating tables according to the invention comprise a substructure for supporting the stretcher, wherein the substructure comprises a receiving space for the arm. The substructure including the receiving space typically enables the arm to be completely received or to be completely retracted. In this manner, the arm may be hidden completely, i.e. stored below the supporting surface so that undisturbed operating is possible. In typical embodiments the receiving space is associated with a parking position of the arm. In typical arms which are displaceable along a longitudinal direction of the stretcher, at least one parking position will be typically provided which is associated with the receiving space in the substructure, so that it is possible to completely hide the arm in the substructure at the parking position. In this case “to completely hide” typically means that the arm does no longer protrude over the stretcher's supporting surface by any component. A parking position is realized in further embodiments by a kinematic mechanism which enables the arm to be folded, for example downwards or to the side. Such a kinematic mechanism may be realized in an actuator which also serves the purpose of displacing the arm lengthwise.

In typical embodiments, the operating table comprises a second arm having second X-ray tubes. The arms are typically arranged on the same level in the longitudinal direction. Typically, the arms are kinematically coupled to one another and, if necessary, to the detector, so that an axial displacement of the parts along the strecher's longitudinal direction is simultaneously possible. When two arms are provided, advantages are seen in that the individual arm may be configured to be shorter, so that the corresponding receiving space as well may be shorter. In further embodiments a plurality of arms is arranged on different transverse planes of the stretcher. In this case, also arranged in the respective transverse planes are detectors which in turn are typically displaceable lengthwise together with the arms. This offers the advantage that a plurality of transverse planes can be captured simultaneously.

Typically, the arm in an extended position, in which it is arranged partially in an area above the supporting surface, is still movable by an angle of at least 10°, at least 20° or at least 30° during the operation of the X-ray tube or X-ray tubes. This facilitates computer tomographies to be taken. Typically, the arm in the extended state may be moved at least by an angular range corresponding to the angular distance between two X-ray tubes of the arm. In this way, every angular range along the arm may be scanned by X-ray tubes. In the case of X-ray tubes arranged, for example, at a spacing of 30°, the arm is correspondingly movable typically by at least 30° in its angular position.

Typical X-ray tubes used in embodiments of the invention exhibit a size of at most 100 mm, typically at most 70 mm or typically at most 50 mm. “Size” designates in this case the largest extension, typically the length. Typical X-ray tubes used in operating tables of this invention exhibit a diameter of a maximum of 20 mm, a maximum of 15 mm or typically a maximum of 12 mm. This enables the X-ray tubes to be positioned in the arm. Typical dimensions of the arm are a width of a maximum of 50 cm or a maximum of 30 cm or a thickness of a maximum of 10 cm or a maximum of 7 cm. This enables a particularly compact design. Typical X-ray tubes feature an accelerating voltage of at least 50 kV, typically an accelerating voltage of at least 70 kV that is possible when in operation.

In typical embodiments, the operating table comprises a targeting system. The targeting system is typically fastened to the arm. Another option for the targeting system is to be provided on an additional holder mounted to the operating table. In typical embodiments, the targeting system is associated with the control device. In this way it is possible to focus on structures by means of the targeting system which can be depicted in computer tomography. The targeting system typically comprises a laser or another source of light. In this way, piercing sites may be marked for injections, for example.

Typical embodiments of operating tables according to the invention comprise an opening in the arm typically for passing an injection means or an endoscope. The targeting system is typically fastened to the arm. The targeting system is in this case typically configured to point into an area below the opening so that a targeted injection or targeted introduction of an endoscope through the opening will be possible by means of the targeting system.

### BRIEF DESCRIPTION OF THE DRAWING

Further advantages and features of preferred embodiments of the invention will be explained below using the attached drawings, wherein the figures show:

FIG. 1 is a schematic cross-sectional view of a typical embodiment of an operating table;

FIG. 2 is a further schematic cross-sectional view of the embodiment of FIG. 1 in another operating state with the arm extended;

FIG. 3 is a top view of the embodiment of FIGS. 1 and 2; and

FIG. 4 is a schematic sectional view of a further embodiment of an operating table.

### DESCRIPTION OF PREFERRED EMBODIMENTS

Typical embodiments will be described below with reference to the figures, wherein the invention is not restricted to the exemplary embodiments, the scope of the invention being rather determined by the claims.

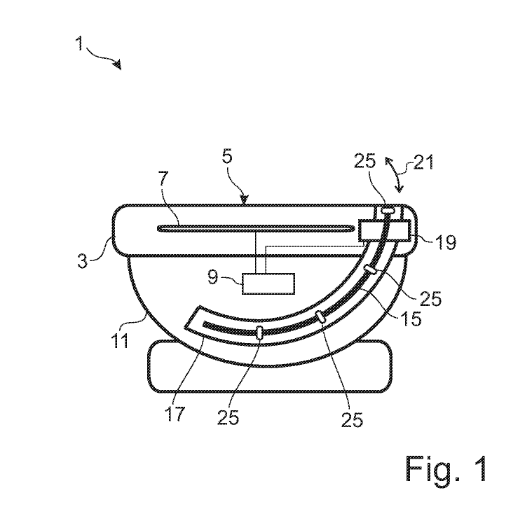

FIG. 1 shows a typical embodiment of an operating table 1 in a schematic cross-sectional view. The operating table 1 comprises a stretcher 3 which is provided to receive a patient. For this purpose, the stretcher 3 exhibits a supporting surface 5 on its upper side. Within the stretcher 3 and below the supporting surface 5 a detector 7 is arranged. The detector 7 is adapted to detect both X-rays, for example for a computer tomography, and gamma rays within the scope of a PET scan or PET/CT scan.

Typical detectors of embodiments are adapted to detect X-rays or gamma rays within the scope of PET, SPECT or PET/CT scans. In further exemplary embodiments, the detector is adapted to detect only X-rays or merely gamma rays. This may cause the detector to be of lower cost.

Detectors which are capable of detecting different kinds of rays, for example X-rays and gamma rays, are suitable for a more universal use.

The detector 7 is associated with a control device 9 which is located within a substructure 11 of the operating table 1. The control device 9 is configured to receive and process signals from the detector 7. Furthermore, the control device provides the high voltage for X-ray tubes in typical embodiments.

Processed data of the detector 7 may be used to generate images of a conventional or radioscopic X-ray scan, computer tomography or a PET image. Further evaluations are possible in embodiments, for example SPECT. For this purpose, the control device 9 may be connected to a monitor.

In embodiments, additional detectors may also be integrated in the receiving space or in the arms.

The operating table 1 of the embodiment of FIG. 1 comprises an arm 15. During surgery or in a storing or parking state, the arm 15 is usually arranged in a receiving space 17 of the operating table's 1 substructure 11. When the arm 15 is received within the receiving space 17, it is situated below the supporting surface by a major part of its length. In typical embodiments such as shown in FIG. 1 for instance, the arm 15 may be retracted completely into an area below the supporting surface 5.

By means of an actuator 19 which is connected to the control device 9 to receive control commands from the control device 9, the arm 15 may be extended into an area above the supporting surface 5 by at least a major part. The arm's 15 moving direction in this case follows the double arrow referenced 21 in FIG. 1. The actuator 19 is moreover adapted to move the arm 15 along a longitudinal direction, thus perpendicular to the drawing plane of FIG. 1, along a linear guide (not shown in FIG. 1). This is in particular possible in the extended state of the arm 15. Extended state means in this case that the arm 15 is positioned in an area above the supporting surface 5 at least by a major part.

Integrated into the arm are X-ray tubes 25 which are distributed over the arm 15 for instance at an equidistant angular distance of 30°. The X-ray tubes, for example, are of the carbon nanotubes (CNT) type. In further exemplary embodiments, other X-ray tubes are used, wherein exemplary embodiments can be designed in a particularly advantageous manner with small dimensions of the arm if small X-ray tubes are used which have a maximum overall length of 7 cm or a maximum of 5 cm. The cross-section of FIG. 1 corresponds to a parking position of the arm 15. The arm's 15 parking position corresponds to the position in which the arm can be hidden in the receiving space 17. Such a parking position has the advantage that the operating table's substructure is not required to have a slot over its entire length, but only at the parking position, for receiving the arm 15. In further exemplary embodiments, the arm is movable in the longitudinal direction even when in the retracted position. This requires the substructure to be constructed in a corresponding way, however, enabling the arm to be extended in various places of the operating table.

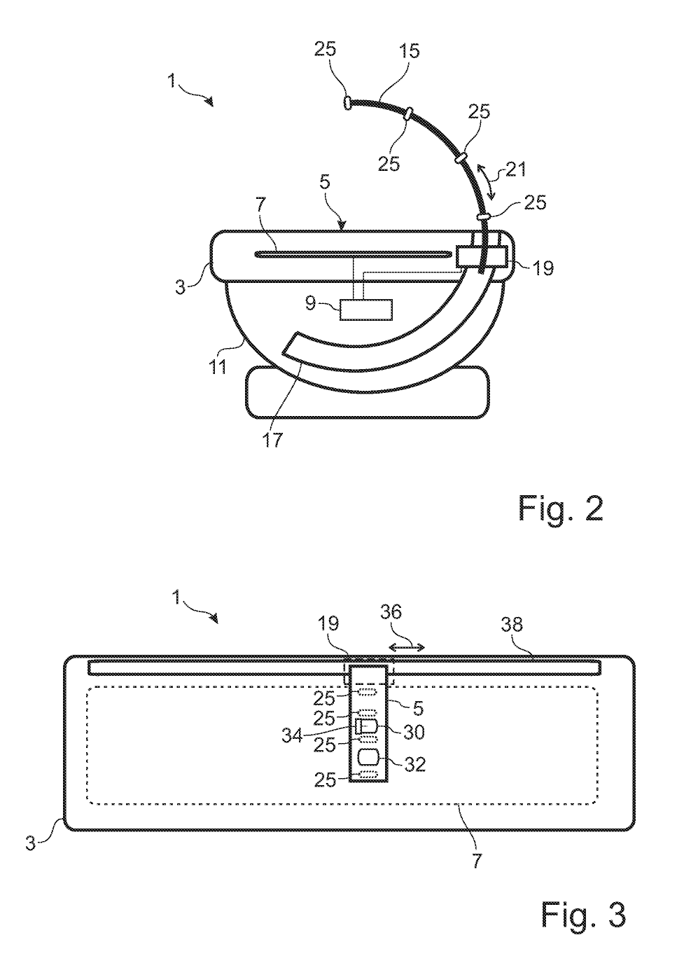

FIG. 2 shows the exemplary embodiment of FIG. 1 again, wherein a position of the arm 15 is shown in FIG. 2 in which the arm is positioned above the supporting surface 5 of the stretcher 3 by a major part.

The arm exhibits typically a radius of 40 cm. The state in which the arm is arranged above the supporting surface is also referred to as the arm's extended state.

In the extended state, it is possible to irradiate a patient situated on the supporting surface 5 with X-rays by means of the X-ray tubes 25. The X-rays are detected by the detector 7 in the stretcher 3 depending on the absorption in the patient's tissue. By moving the arm along the double arrow 21, the X-ray tubes 25 can be brought in different positions so that a computer tomography can be produced.

In the arm's 15 extended state, it is moreover possible to move the arm 15 by means of the actuator 19 along the longitudinal direction of the stretcher 3. This is schematically represented in FIG. 3 where the embodiment of FIGS. 1 and 2 is schematically shown in a top view. In FIG. 3, an opening 30 in the arm 15 and a monitor 32 are visible which is attached to the arm 15. By means of the opening 30, it is possible to introduce for instance an endoscope into the patient through the arm 15.

Embodiments comprise an arm that is extendable and movable in the circumferential direction, the movements of which may also be utilized to move the at least one X-ray tube in various angular positions. In further embodiments, the arm comprises a drive to move the at least one X-ray tube relative to the arm in the circumferential direction, for example, by at least 45° or by at least 90°. Thus, different positions may be quickly reached with a single X-ray tube or a plurality of X-ray tubes may be quickly displaced at the same time.

In the area of the opening, a targeting system 34 is moreover positioned which is likewise connected to the control device 9. Through the targeting system 34 structures can be focused which had been identified before by means of computer tomography on the operating table 1 using the detector 7 and the control device 9 and the monitor 32. In this manner, a targeted introduction of for instance an endoscope is possible. The targeting system 34 comprises in typical embodiments a laser or another source of light such as a halogen lamp capable of marking a certain point on the patient's body surface.

FIG. 3 also shows how the arm 15 can be moved along an arrow 36 in the longitudinal direction of the operating table 1. To this end, the actuator 19 (FIGS. 1 and 2) is used which runs along a linear guide 38. In further embodiments, a second arm is provided at another position in the longitudinal direction of the operating table 1 to be able to produce computer tomographies on several transverse levels, for example.

The detector 7 of the exemplary embodiment of FIGS. 1 to 3 is kinematically coupled to the arm 15 via the actuator 19 so that, in case of a longitudinal displacement of the arm 15, the detector 7 will be displaced simultaneously therewith in the longitudinal direction. This enables CT images each in different transverse planes of a patient who is supported on a stretcher of exemplary embodiments. The connections from the control device to the arm or to the detector of embodiments are routed via the linear guide or are wireless, an energy supply being merely performed via the linear guide in this case. In this manner, a maximum of two poles need to be transferred so that costly multi-pole wirings can be omitted. Even in case of a completely wired transfer along the linear guide, the option is given to route a cable in the linear guide in such a manner that a displacement of the actuator together with the arm and the detector is enabled.

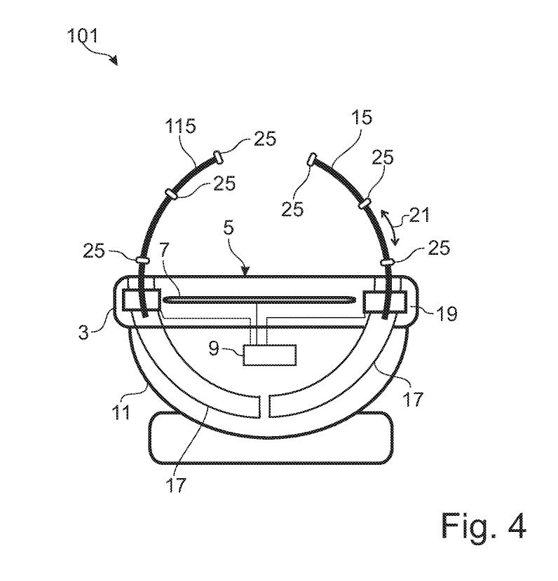

FIG. 4 shows a further embodiment in which two arms 15 and 115 are present. The arms 15 and 115 are arranged in a transverse plane together with a detector 7 and can be moved commonly in the longitudinal direction of the operating table 101 in embodiment 4. In the description of FIG. 4 identical or similar parts to those in the descriptions of FIGS. 1 to 3 will not be explained again individually in all details or as a whole. Identical reference numerals usually refer to identical or similar parts.

Exemplary embodiments including a second arm offer the advantage that the arms may each be designed to be shorter so that receiving spaces in a substructure of the operating table can be designed to be smaller, for example. Furthermore it is possible to cover a larger area with two arms under certain circumstances.

Like the exemplary embodiments of FIGS. 1 to 3, the exemplary embodiment of FIG. 4 as well comprises a control device 9 and for instance monitors or openings in the arms 15 and 115 which, however, are not illustrated in FIG. 4 for the sake of simplicity.

In typical embodiments two arcuate arms and a detector arranged in the stretcher are present. The detector is in this case typically designed to be flat. In further embodiments curved detectors are arranged in a substructure of the operating table. This can result in advantages when taking a computer tomography since rays are not required to be detected in an acute angle by the detector.

In further exemplary embodiments the arms or the arm are or is foldable about a longitudinal axis of the operating table so that the distance of the arm from the patient may be varied or the adaptation of the arm may be adjusted to patients having different girths. Such an embodiment can be realized, for example, in that an actuator, which is present in any case, in addition enables the arm to be tilted about the longitudinal axis in the extended state of the arm. In this case, it must be considered that the kinematic restriction of the receiving space, in which the arm is received, is only given when the arm is retracted. In contrast, tilting of the arm about a longitudinal axis is possible when the arm is extended.

Typically, actuators of exemplary embodiments are configured to transfer the arm in less than 15 seconds or in less than 10 seconds form an extended state to a retracted state. This has the advantage that intraoperative computer tomographies can be quickly generated and the arm can also quickly hide away again.

In embodiments, at least one further detector is integrated in the arm or the substructure. A wide coverage can thus be achieved. The detector may be positioned, for example, in a curved form in one of the arms. A further option is the positioning in a substructure, with the stretcher or parts of the stretcher preferably exhibiting low radiation absorption.

1. An operating table including
a stretcher for receiving a patient on a supporting surface of the stretcher,
a detector which is arranged below the stretcher's supporting surface,
an arcuate arm which is extendable from an area below the supporting surface at least partially into an area above the supporting surface, and
an X-ray tube positioned in the arm.

2. The operating table according to claim 1, wherein the arcuate arm has a shape with a radius of a maximum of 50 cm.

3. The operating table according to claim 1, wherein at least two or at least five X-ray tubes are positioned in the arm.

4. The operating table according to claim 3, wherein the X-ray tubes are arranged to be evenly distributed over the arm.

5. The operating table according to claim 1, wherein the arm is displaceable relative to the stretcher in a longitudinal direction of the stretcher.

6. The operating table according to claim 5, wherein the arm is kinematically coupled with the detector in the longitudinal direction of the stretcher, so that the arm and the detector are simultaneously displaceable along the longitudinal direction.

7. The operating table according to claim 1, including a substructure for mounting the stretcher, wherein the substructure comprises a receiving space for the arm.

8. The operating table according to claim 7, wherein the receiving space is assigned a parking position of the arm.

9. The operating table according to claim 1, wherein the arm is supported in a linear guide of the operating table.

10. The operating table according to claim 1, including a second arm having further X-ray tubes.

11. The operating table according to claim 1, wherein the arm comprises a monitor which is connected to a control device.

12. The operating table according to claim 1, including a targeting system.

13. The operating table according to claim 1, wherein the arm comprises an opening for passing an injection means or an endoscope.

14. The operating table according to claim 1, wherein at least one further detector is integrated in the arm.

15. The operating table according to claim 7, wherein at least one further detector is integrated in the substructure.
